(19) United States
(12) Patent Application Publication
(10) Pub. No.: US 2017/0241979 A1
(43) Pub. Date: Aug. 24, 2017

(54) POTENCY ASSAY FOR THERAPEUTIC AGENTS

(71) Applicant: OPEXA THERAPEUTICS, INC., The Woodlands, TX (US)

(72) Inventors: Don HEALEY, Anderson, TX (US); Lauren West COLLISON, The Woodlands, TX (US)

(21) Appl. No.: 15/113,612

(22) PCT Filed: Jan. 23, 2015
(86) PCT No.: PCT/US2015/012776

§ 371 Date: Jul. 22, 2016

Related U.S. Application Data
(60) Provisional application No. 61/931,470, filed on Jan. 24, 2014.

(51) Int. Cl. G01N 33/50

(52) U.S. Cl. CPC G01N 33/505; G01N 33/5052; G01N 2333/70596; G01N 2500/10

(57) ABSTRACT

Provided herein are methods of determining the tolerogenic potential of a therapeutic agent comprising determining the ability of the therapeutic agent to increase the expression of tolerogenic markers, e.g., PD-L1 , by antigen presenting cells such as monocytes, macrophages, B cells and dendritic cells.

### FIELD OF INVENTION

The present invention provides methods of determining the tolerogenicity and/or potency of a therapeutic agent.

### BACKGROUND OF THE INVENTION

Presently, different test methods, such as assays of physiochemical properties, antigenicity, immunogenicity, infectivity and protection against infection or disease, are used to measure the potency of a therapeutic agent. The application depends on the nature of the therapeutic agent and the purpose of the test. For example, in the case of vaccines, the potency is often determined by measuring the immune response in the target animal species or in another species, e.g. mice or rats.

Previous reports indicate that T cell immunotherapy of autoimmune diseases, e.g., vaccination with attenuated autoreactive T cells, induce a regulatory immune response to control the aberrant autoimmune responses by increasing anti-idiotypic and/or anti-ergotypic activity towards the autoreactive T cells. It is thought that administration of self-antigen specific T cells can elicit unique paracrine signals that facilitate a tolerogenic network of antigen presenting cells (APCs) and/or effector T cells to help control the inflammation associated with the autoimmune disorder. As such, measuring the potency of such T cell based therapeutic agents requires measuring the induction of a regulatory immune response.

High-throughput, preferably in vitro, assays capable of measuring the potency of a therapeutic agent, particularly therapeutic agents that induce regulatory immunity, are needed.

### SUMMARY OF INVENTION

Disclosed herein is a method of assessing the potency of a therapeutic agent, the method comprising determining the tolerogenicity of the therapeutic agent. Methods of determining the tolerogenicity of a therapeutic agent generally comprises the steps of contacting the therapeutic agent with a test sample comprising test antigen presenting cells, preferably in vitro; measuring expression of at least one tolerogenic marker by the test antigen presenting cells contacted with the therapeutic agent; and comparing the expression of the at least one tolerogenic marker by the test antigen presenting cells with an appropriate control; wherein an increase in expression of the at least one tolerogenic marker by the test antigen presenting cells compared to the control antigen presenting cells determines that the therapeutic agent is tolerogenic. In preferred embodiments the at least one tolerogenic marker comprises a PD-1 ligand, e.g. PD-L1. In preferred embodiments, the measuring step is performed within about 24-72 hours after the contacting step, still more preferably within about 48-72 hours, and most preferably within about 72 hours. In one embodiment, the potency of the therapeutic agent is directly correlated with the tolerogenicity of the therapeutic agent.

In one embodiment, the therapeutic agent is administered to a patient in need thereof to tolerize the patient to a particular agent, e.g., downregulate the patient's immune response to that antigen, such as an allergen or autoantigen. In one embodiment, the therapeutic agent is selected from the group consisting of an allergen, a tolerizing epitope thereof, a vaccine comprising immune cells specific to the allergen, a vaccine comprising fragments of immune cells specific to the allergen, and a nucleic acid encoding the allergen or tolerizing epitope thereof. Preferably, the therapeutic agent is selected from the group consisting of an autoantigen, a tolerizing epitope thereof, a vaccine comprising immune cells specific to the autoantigen, a vaccine comprising tolerizing fragments of immune cells specific to the autoantigen, and a nucleic acid encoding the autoantigen or tolerizing epitope thereof

In an exemplary preferred embodiment, the therapeutic agent comprises T cells specific to an autoantigen, wherein the autoantigen is preferably selected from the group consisting of a myelin protein (e.g., myelin basic protein, proteolipid protein, myelin oligodendrocyte protein), aquaporin 4, and platelet membrane glycoproteins IIb-IIIa and Ib-IX. Most preferably, the therapeutic agent comprises attenuated T cells specific to a myelin protein. In another embodiment, the T cells and the test and control antigen presenting cells are allogeneic. In certain preferred embodiments, the T cells and the test and control antigen presenting cells are autologous.

In preferred embodiments, the test and/or control samples comprising antigen presenting cells comprise monocytes, dendritic cells, B cells and/or cell lines derived from monocytes, macrophages, dendritic cells, or B cells. In some embodiments, the antigen presenting cells comprise a cell line derived from monocytes, e.g., U937, THP-1, etc. Preferably, the test and control antigen presenting cells comprise B cells. Most preferably, the test and control antigen presenting cells comprise monocytes.

In one embodiment, the at least one tolerogenic marker is measured in combination with at least one marker of an antigen presenting cell, such as CD86. More preferably, a high throughput method of detecting the tolerogenic marker, and optionally the antigen presenting cell marker, is used. Most preferably, flow cytometric analysis is used to simultaneously detect the cell surface expression of the at least one tolerogenic marker, e.g., PD-L1, and the antigen presenting cell marker, preferably CD86, on the antigen presenting cells.

Also provided herein are kits to determine the tolerogenicity of a therapeutic agent, the kits comprising an antibody to a tolerogenic marker and an antibody to an antigen presenting cell marker. Preferably, the kit may also comprise detection reagents for detecting the antibody or antibodies and/or instructions for using the kit.

### BRIEF DESCRIPTION OF THE DRAWINGS

FIG. 1 shows flow cytometric dot plots showing cell surface PD-L1 expression (y-axis) by autologous lymphocytes (CD86⁻, x-axis) or autologous monocytes (CD86; x-axis) when incubated (A) alone or (B) in the presence of TCELNA®.

FIG. 2 shows the percentage of monocytes expressing PD-L1 on the cell surface (y-axis) when incubated alone (▪) or in the presence of autologousT cells (□) from five independent TCELNA® products (x-axis).

FIG. 3 shows the fold change in mean fluorescence intensity (MFI; y-axis) of monocytes bound with fluorescent anti-PD-L1 antibody after incubation with autologous T cells from five independent TCELNA® products (x-axis).

FIG. 4 shows the percentage of immune cells expressing PD-L1 on the cell surface (y-axis) when incubated for (A) 24 hours or (B) 72 hours alone or with T cells from an allogeneic TCELNA® product.

### DETAILED DESCRIPTION

It is believed that antigen presenting cells (APCs), particularly professional antigen presenting cells, upon interaction with irradiated, apoptotic cells (such as attenuated autoreactive T cells) are capable of undergoing significant phenotypic and tolerogenic changes. Following exposure with irradiated, autologous leukocytes, dendritic cells demonstrate tolerogenic characteristics including enhanced secretion of IL-10 and impaired stimulation of naïve T cell proliferation (Zheng D H et al. (2010) Biochem Biophys Res Commun. 395(4):540-6). Moreover, chronic viral infection with HIV results in increased expression of PD-L1 on various cell types, IL-10 secretion from monocytes, and suppression of T cell expansion (Said E A et al. (2010) Nat Med. 16(4):452-9). T cell expansion and function are restored by blocking interactions between, e.g., PD-L1 and PD-1 and between IL-10 R and IL-10 , suggesting that interactions involving PD-L1 and/or IL-10 , their respective receptors and other moleucles in the respective pathways may be useful markers of tolerance induction.

As used herein, a tolerogenic marker may comprise a protein for which the upregulated expression thereof may be immediately detected by antigen expression cells of contact with a tolerizing agent. For example, the expression of a tolerogenic marker comprising a PD-1 ligand, preferably PD-L1 , may be detected within 24 hours, 48 hours, or 72 hours, preferably within 48 hours, and most preferably within 24 hours after contact with a tolerizing agent.

In multiple sclerosis, reduced APC expression of PD-L1 and enhanced secretion of proinflammatory cytokines correlates with progression in MS patients (Karni A. et al. (2006) J Immunol. 177(6):4196-202). Conversely, treatment of MS with IFN-β therapy strongly enhances PD-L1 expression on monocytes and DCs and significantly inhibits autologous CD4 T cell activation (Schreiner B (2004) J Neuroimmunol. 155:172-182.).

TCELNA® is a T-cell immunotherapy for Multiple Sclerosis (MS) which is composed of a pool of autologous myelin-reactive T cells, expanded from an individuals' peripheral blood mononuclear cells. As disclosed herein, the potency of the TCELNA® product may be tested by monitoring PD-L1 expression by antigen presenting cells in response to TCELNA® exposure. Specifically, in response to culture with the TCELNA® product, PD-L1 expression is induced in responder monocytes.

These above observations support the detection of expression of a tolerogenic marker, e.g., a tolerogenic marker comprising a PD-1 ligand, e.g., PD-L1 , by antigen presenting cells as a means to determine the tolerogenicity of a therapeutic agent.

Therapeutic Agents

Accordingly, in one aspect, the invention provides methods for determining the tolerogenicity of a therapeutic agent. As used herein, the term “tolerogenicity” refers to the ability of a therapeutic agent to “tolerize” or induce “tolerance” to a particular antigen. The term “tolerance” refers to a reduction in the adaptive immune response, e.g., the T cell and/or antibody response, to a specific antigen. The reduction in the immune response to the specific antigen may be concomitant with increased sensitization and/or response of special subsets of immunoregulatory cells, e.g., anti-idiotypic and/or anti-ergotypic immune cells.

A skilled artisan will recognize that the tolerogenicity of a therapeutic agent may be associated with the potency of the therapeutic agent. “Potency” as used herein refers to the ability of a therapeutic agent to produce a desired effect after administration.

In preferred embodiments, the desired effect of a therapeutic agent is tolerization of the immune response to a particular antigen, e.g., an allergen, autoantigen. In these embodiments, the tolerogenicity of a therapeutic agent is directly correlated with its potency. In preferred embodiments, the methods disclosed herein are used to determine the potency of a therapeutic agent that seeks to induce tolerance to an allergen or an autoantigen.

“Allergens” are any substances that can cause an undesired (e.g., a Type 1 hypersensitive) immune response (i.e., an allergic response or reaction) in a subject. Allergens include, but are not limited to, plant allergens (e.g., pollen, ragweed allergen), insect allergens, insect sting allergens (e.g., bee sting allergens), animal allergens (e.g., pet allergens, such as animal dander or cat Fel d 1 antigen), latex allergens, mold allergens, fungal allergens, cosmetic allergens, drug allergens, food allergens, dust, insect venom, viruses, bacteria, etc. Food allergens include, but are not limited to milk allergens, egg allergens, nut allergens (e.g., peanut or tree nut allergens, etc. (e.g., walnuts, cashews, etc.)), fish allergens, shellfish allergens, soy allergens, legume allergens, seed allergens and wheat allergens. Insect sting allergens include allergens that are or are associated with bee stings, wasp stings, hornet stings, yellow jacket stings, etc. Insect allergens also include house dust mite allergens (e.g., Der P1 antigen) and cockroach allergens. Drug allergens include allergens that are or are associated with antibiotics, NSAIDs, anaesthetics, etc. Pollen allergens include grass allergens, tree allergens, weed allergens, flower allergens, etc. “Allergens associated with an allergy” are allergens that generate an undesired immune response that results in, or would be expected by a clinician to result in, alone or in combination with other allergens, an allergic response or reaction or a symptom of an allergic response or reaction in a subject.

“Autoantigens” are normal tissue constituents in the body targeted by an autologous humoral (B cell) or T cell mediated immune response that often results in damage to the tissue and/or autoimmune disease. “Autologous” as used herein refers to cells or tissues derived from the same individual or cells or tissues that are immunologically compatible, e.g., have an identical MHC/HLA haplotypes. “Allogeneic” as used herein refers to cells or tissues that are genetically dissimilar and immunologically incompatible, e.g., have differing MHC/HLA haplotypes. Nonlimiting examples of autoantigens include constituents of myelin protein (e.g., myelin basic protein, proteolipid protein, myelin oligodendrocyte protein), aquaporin 4, platelet membrane glycoproteins IIb-IIIa and Ib-IX, insulin, proinsulin, glutamic acid decarboxylase (GAD), GAD65, GAD67, heat-shock protein 65 (hsp65), islet-cell antigen 69 (ICA69), islet cell antigen-related protein-tyrosine phosphatase (PTP), GM2-1 ganglioside, Tep69, an islet-cell protein tyrosine phosphatase and the 37-kDa autoantigen derived from it (including IA-2), phogrin, human chondrocyte glycoprotein-39, collagen, collagen type II, cartilage link protein, ezrin, radixin, moesin, mycobacterial heat shock protein 6, desmoglien, β-2-GPI, Ku (p70/p80) autoantigen or its 80-kd subunit protein, the nuclear autoantigens La (SS-B) and Ro (SS-A), proteasome β-type subunit C9, the centrosome autoantigen PCM-1, polymyositis-scleroderma autoantigen (PM-Scl), autoantigen CENP-A, U5, the nucleolar U3- and Th(7-2) ribonucleoproteins, the ribosomal protein L7, hPop1, a 36-kd protein from nuclear matrix antigen, thyroid peroxidase and the thyroid stimulating hormone receptor, the human TSH receptor, acetylcholine receptor, muscular receptor kinase, or any other suitable autoantigen.

A skilled artisan will readily recognize therapeutic agents that seek to induce tolerance to an allergen or an autoantigen include, but are not limited to, the full-length allergen or the full-length autoantigen; tolerizing epitopes of the allergen or tolerizing epitopes of the autoantigen,; vaccines comprising immune cells, which may be attenuated, that are specific for or responsive to the allergen or autoantigen; vaccines comprising tolerizing fragments of immune cells that are specific for or responsive to the allergen or autoantigen (e.g., T cell receptors, B cell receptors, derivatives thereof etc.), and vaccines comprising nucleic acids, e.g., DNA or RNA, encoding the allergen, autoantigen, tolerizing epitopes thereof, and tolerizing fragments of immune cells specific for and/or responsive to the allergen or autoantigen. As used herein, a tolerizing epitope of an allergen or autoantigen may include but is not limited to, fragments, fusion proteins, peptide mimeotypes, and altered peptides of the allergen or autoantigen, respectively.

“Epitope”, also known as an antigenic determinant, is the part of an antigen, e.g., allergen, autoantigen, that is recognized by the immune system, specifically by, for example, antibodies, B cells, or T cells. Epitopes are often presented to immune cells by MHC or HLA molecules found on nucleated cells. In some embodiments, the epitope itself is an antigen.

In one embodiment, the therapeutic agent seeks to induce tolerance to an autoantigen. In preferred embodiments, the autoantigen is selected from the group consisting of a myelin protein, aquaporin-4, and platelet membrane glycoproteins IIb-IIIa and Ib-IX. In a more preferred embodiment, the autoantigen is a myelin protein selected from the group consisting of myelin basic protein, proteolipid protein, and myelin oligodendrocyte protein. Preferably, the therapeutic agent that seeks to induce tolerance to an autoantigen comprises attenuated T cells, wherein the T cells are specific to the autoantigen.

In one embodiment, the desired effect of the therapeutic agent is activation of the immune response to a particular antigen. It is well-known in the art that such therapeutic agents include pathogenic antigens, tumor antigens, fragments of pathogenic antigens, fragments of tumor antigens, nucleic acids encoding pathogenic antigens, and nucleic acids encoding tumor antigens. Non-limiting examples of well-known tumor antigens include cytokeratins, particularly cytokeratin 8, 18 and 19, as an antigen for carcinomas; epithelial membrane antigen (EMA); human embryonic antigen (HEA-125); human milk fat globules, MBr1, MBr8, Ber-EP4, 17-1A, C26 and T16; desmin and muscle-specific actin as antigens of myogenic sarcomas; placental alkaline phosphatase, beta-human chorionic gonadotropin, and alpha-fetoprotein as antigens of trophoblastic and germ cell tumors; prostate specific antigen as an antigen of prostatic carcinomas; carcinoembryonic antigen of colon adenocarcinomas; HMB-45 as an antigen of melanomas; and chromagranin-A and synaptophysin as antigens of neuroendocrine and neuroectodermal tumors. In these embodiments, the tolerogenicity of a therapeutic agent is not directly correlated with the potency of the therapeutic agent, and instead, the tolerogenicity of the therapeutic agent may be inversely correlated with its potency.

Antigen presenting cells, tolerogenic markers, and measuring same

As disclosed herein, the methods of determining the tolerogenicity of a therapeutic agent disclosed herein generally comprise a contacting the therapeutic agent with antigen presenting cells and measuring the expression of at least one tolerogenic marker by antigen presenting cells.

As discussed above, epitopes are often presented to immune cells by MHC or HLA molecules found on all nucleated cells. In other words, all nucleated cells are capable of antigen presentation using class I molecules. However, some epitopes are presented to immune cells by MHC or HLA molecules found on professional antigen-presenting immune cells, such as on macrophages, monocytes, B cells, dendritic cells, and cell lines derived therefrom. In a preferred embodiment, the methods disclosed herein comprises measuring the expression of a tolerogenic marker by professional antigen presenting cells. In preferred embodiments, expression of a tolerogenic marker by antigen presenting cells comprising monocytes, macrophages, B cells, or cell lines derived therefrom is measured. More preferably, the expression of a tolerogenic marker by antigen presenting cells comprising macrophages, monocytes, and/ or cell lines derived therefrom, e.g., the U937 or THP-1 cell line, most preferably monocytes, is measured.

Studies comparing the phenotype of antigen presenting cells (APCs) from individuals with secondary progressive multiple sclerosis (SP-MS) to individuals with relapsing-remitting multiple sclerosis (RR-MS) or healthy controls suggests that APCs from individuals with autoimmune diseases are biased toward an inflammatory response, marked by reduced PD-L1 expression and enhanced secretion of inflammatory mediators (Karni A et al. (2006) J. Immunol. 177(6):4196-202.). However, treatment with IFN-β therapy strongly enhances PD-L1 expression on monocytes and DCs and renders DCs more tolerogenic/regulatory, supported by their failure to induce autologous CD4 T cell activation upon secondary culture (Schreiner B et al (2004) J. Neuroimmunol. 155: 172-182.). Taken together, these data suggest that induction of PD-L1 expression in SP-MS patients may help to restore the tolerogenic/regulatory network that is dysfunctional in SP-MS patients, revealing a novel therapeutic aim. As disclosed herein, PD-L1 expression is strongly induced in monocytes as a result of exposure to TCELNA®, suggesting that the induction of tolerogenic markers by antigen presenting cells can serve as indication of both product potency as well as mechanism of action.

Accordingly, in one embodiment, the expression of a PD-1 ligand, e.g., PD-L1 , PD-L2, etc. as a tolerogenic marker is measured. In one embodiment, the expression of PD-L1 by an antigen presenting cell is measured. In other embodiments, the expression of a PD-1 ligand is measured within 72 hours after contacting the antigen presenting cells with a therapeutic agent. Preferably, the expression of a PD-1 ligand by antigen presenting cells is measured about 24 hours after contacting the antigen presenting cells with a therapeutic agent. There are a variety of assay formats known to those of ordinary skill in the art for detecting the expression of a tolerogenic marker by antigen presenting cells. See, e.g., Harlow and Lane, Antibodies: A Laboratory Manual, Cold Spring Harbor Laboratory, 1988. As nonlimiting examples, detection of a tolerogenic marker protein may be performed using a antibody that binds to the protein and which is preferably detectably labeled (e.g., with a radioactive label, a luminescent label, a fluorescent label, an enzyme, etc.). The expression of a tolerogenic marker protein by antigen presenting cells may be measured by detecting tolerogenic markers bound by specific antibodies according to well-known methods or assays, e.g. immunoprecipitation, ELISA, Western blot analysis, immunohistochemistry, immunofluorescence, “sandwich” immunoassays, immunoradiometric assays, gel diffusion precipitation reactions, immunodiffusion assays, in situ immunoassays, precipitation reactions, agglutination assays, complement fixation assays, protein A assays, immunoelectrophoresis assays, fluorescence activated cell sorting (FACS) analysis, radioimmunoassay, a strip test, a point of care test, and the like. In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated.

Expression of a tolerogenic marker comprising a cell surface protein, e.g., PD-L1 by antigen presenting cells may be measured by fluorescence activated cell sorting. Alternatively, expression of a tolerogenic marker comprising a cytokine may be measured using a well-known immunoassay, preferably ELISA.

To improve sensitivity, multiple markers may be assayed within a given sample. In particular, one or more other tolerogenic markers may be assayed in combination with markers of antigen presenting cells. Examples of cell surface markers for antigen presenting cells include, but are not limited to, MHC class I, MHC Class II, CD1, CD2, CD3, CD4, CD8, CD11b, CD14, CD15, CD16, CD 19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFN-γ receptor and IL-2 receptor, ICAM-1, Fcγ receptor or other receptor relatively specifically expressed by antigen presenting cells. Examples of cell surface markers for dendritic cells include, but are not limited to, MHC class I, MHC Class II, CD1, CD2, CD3, CD4, CD8, CD11b, CD14, CD15, CD16, CD19, CD20, CD29, CD31, CD40, CD43, CD44, CD45, CD54, CD56, CD57, CD58, CD83, CD86, CMRF-44, CMRF-56, DCIR, DC-ASPGR, CLEC-6, CD40, BDCA-2, MARCO, DEC-205, mannose receptor, Langerin, DECTIN-1, B7-1, B7-2, IFNγ receptor and IL-2 receptor, ICAM-1, Fcγ receptor or other receptor relatively specifically expressed by dendritic cells. Examples of cell surface markers for monocytes/macrophages include, but are not limited to CD14, CD32, CD68, CD115, CD83, p55, CD40 and CD86. Examples of cell surface markers for B cells include, but are not limited to, B220/CD45R, CD19, CD21, CD24, CD37, CD38, CD40, CD80, and CD86.

Combination assays may be done concurrently or sequentially. The selection of markers may be based on routine experiments to determine combinations that results in optimal sensitivity. In preferred embodiments, a cell surface tolerogenic marker and a cell surface marker of an antigen presenting cell are measured simultaneously using fluorescence activated cell sorting and/or flow cytometric analysis. More preferably, cell surface expression of PD-L1 and CD86 are measured simultaneously by flow cytometric analysis.

In preferred embodiments, in measuring the expression of at least one tolerogenic marker by a test sample (i.e., antigen presenting cells contacted with the therapeutic agent), the detected level of expression by the test antigen presenting cells may generally be compared to a detected level of expression by a corresponding and appropriate control sample (i.e., antigen presenting cells, preferably autologous to the test antigen presenting cells, not contacted with a therapeutic agent). In preferred embodiments, a relative increase in the expression level of a tolerogenic marker by test antigen presenting cells and/or in the number of test antigen presenting cells expressing the tolerogenic marker compared to control antigen presenting cells directly correlates with the tolerogenicity and/or potency of the therapeutic agent.

The expression level of the tolerogenic marker by test sample antigen presenting cells and/or the percentage of test antigen presenting cells expressing the tolerogenic marker may be measured in terms of fold increase over the control sample, e.g., a 3-fold increase in detectable expression levels and/or percentage of cells expressing the tolerogenic marker may be considered a tolerogenicity level of 3, a 5-fold increase in detectable expression levels and/or percentage of cells expressing the tolerogenic marker may be considered a tolerogenicity level of 5, a 10-fold increase over the control may be considered an tolerogenicity level of 10, a 100-fold increase over the control may be considered a tolerogenicity level of 100, etc. In one embodiment, a therapeutic agent is determined to be tolerogenic if it's tolerogenicity level is at least about 3, e.g., at least about 5, e.g., at least about 10, e.g., at least about 20, e.g., at least about 100.

Also described herein are kits for determining the tolerogenicity of a therapeutic agent. Such kits typically comprise two or more components necessary for performing a potency assay. Components may be compounds, reagents, containers, instructions and/or equipment. For example, one container within a kit may contain an antibody specific for a tolerogenic marker and an antibody specific for an antigen presenting cell marker. Such kits may also contain a detection reagent as described above that contains a reporter group suitable for direct or indirect detection of antibody binding.

Accordingly, described herein are kits for measuring the expression level of a tolerogenic marker by antigen presenting cells, the kit being useful for providing a result suitable for determining the tolerogenicity and/or potency of a therapeutic agent. A kit may comprise an antibody to a tolerogenic marker, which may optionally be detectably labeled, e.g., with a radioactive label, a luminescent label, a fluorescent label, an enzyme, etc. Methods for detectably labeling proteins are well-known in the art. Such a kit may further include detection reagents specific for markers of antigen presenting cells. Instructions for using the kit for evaluation purposes, including appropriate comparison standards for quantifying and/or evaluating the level of such tolerogenic marker in the context of a particular therapeutic agent, may also be advantageously provided in printed form and/or recorded on a suitable media.

### EXAMPLES

### Example 1

### PBMCS as a Source of Antigen Presenting Cells

### Example 1.1

### Materials and Methods

Responder cells used in the potency assay were obtained from a frozen stock of ficoll separated PBMCs. PBMCs were thawed, washed and resuspended in culture media prior to exposure to TCELNA® product.

The TCELNA® product was taken from liquid nitrogen storage and thawed at 37° C. in a water bath. The sample was irradiated and used immediately upon formulation for the potency assay. Responder PBMCs (1×10⁶) were cultured in polypropylene tubes for 3 days either alone or in combination with TCELNA® (1×10⁶). After 1 or 3 days of culture, PD-L1 , CD95, PD1, CD86, LAG-3, IL-6, IL-4, IL-10 , IL-23, IL-1b, IL-27, IL-21 and/or IL-22 expression by CD14+CD19-CD3-monocytes was monitored by flow cytometry as appropriate. An isotype matched mAb was used to verify specificity of PD-L1 staining (data not shown).

### Example 1.2

### Results

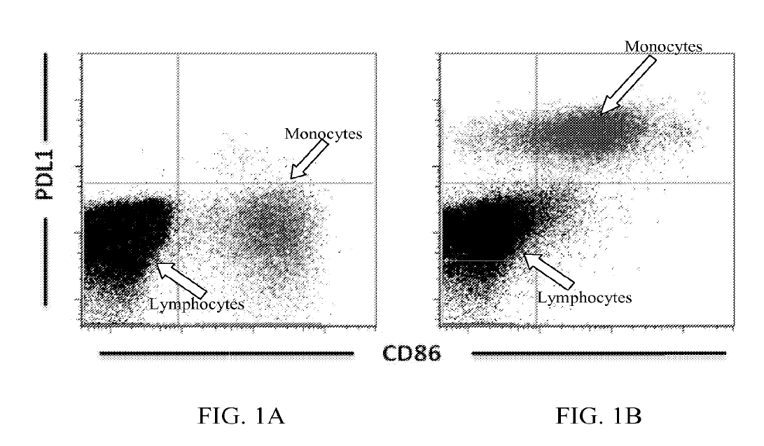

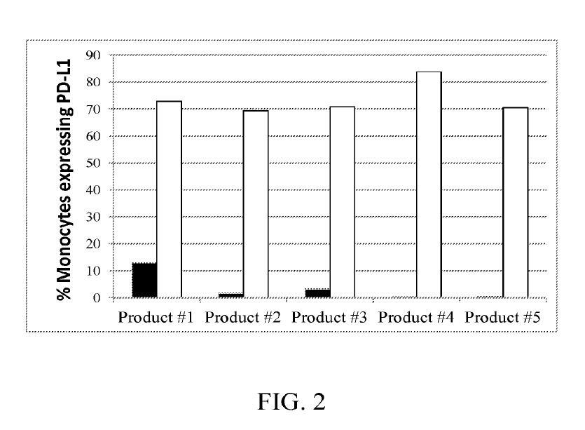

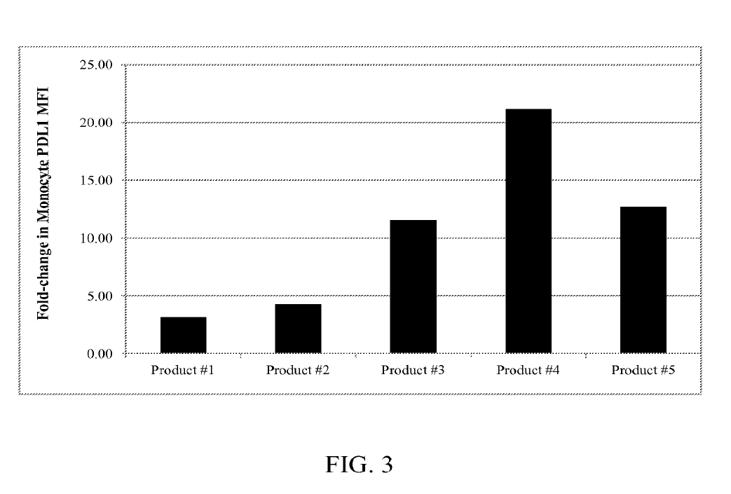

The monocyte based potency assay demonstrates that TCELNA® imparts a tolerogenic/regulatory phenotype upon responder monocytes signified by the induction of PD-L1 expression. Productive modulation of the monocyte phenotype results in PD-L1 expression on >90% of monocytes, as a result of exposure to TCELNA® product. In the data shown, monocytes are identified based on CD14 expression. In the absence of TCELNA®, (PBMCs cultured alone) monocytes fail to express PD-L1 as demonstrated by no more than 15% of cells in the PD-L1 positive fraction (FIGS. 1A and 2). By comparison, culture of PBMCs with TCELNA® product results in at least 60% of monocytes expressing PD-L1 (FIGS. 1B and 2), suggesting potent induction of a tolerogenic/regulatory phenotype. FIG. 2 demonstrates induction of PD-L1 on monocytes following exposure to 5 independent patient products. FIG. 3 shows the fold increase in mean fluorescence intensity by monocytes after incubation with five different TCELNA® products. Taken together, the data suggest that monocytes exposed to the TCELNA® product in vitro undergo fundamental changes in their expression of a regulatory marker, PD-L1 , which may also be indicative of the products mechanism of action in vivo.

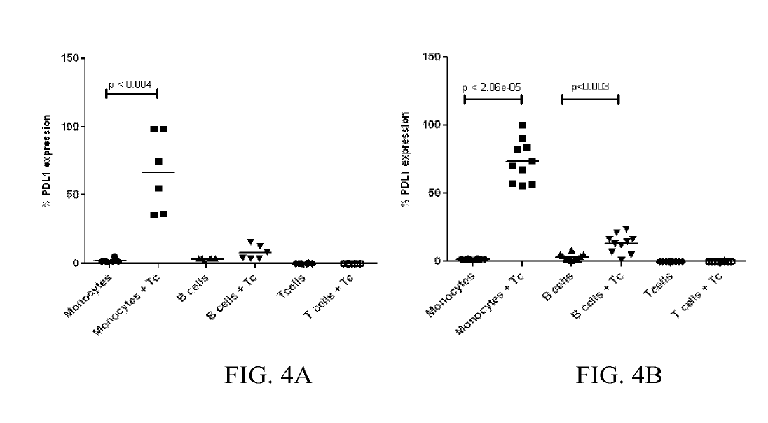

In testing allogeneic antigen presenting cells, formulated and irradiated TCELNA® product was combined with an allogeneic responder PBMC population at a 1:1 ratio. Twenty-four and 72 hours later, the culture was harvested and evaluated using flow cytometry for PD-L1 expression on responder cell populations. FIG. 4 shows the percentage PD-L1 expression of both responder only and responder plus TCELNA® (+Tc) conditions. A Wilcox rank-sum test for paired, independent, samples demonstrated a significant upregulation of PD-L1 expression on monocytes at both 24 and 72 hours (p<0.004 and p<0.00002, respectively), see, FIG. 4A, and FIG. 4B, respectively. A similar analysis of PD-L1 expression on B cells and T cells also shows a significant induction of PD-L1 expression at 72 hrs of culture in the B cell population (p<0.003), but no induction of PD-L1 by T cells at either time points.

Cell surface expression of PD-L1 , CD95, PD1, CD86, LAG-3 and cytokine expression of IL-6, IL-4, IL-10 , IL-23, IL-1b, IL-27, IL-21 and IL-22 by monocytes was analyzed about 24 hours after incubation with irradiated TCELNA® product. Of the markers tested, only PD-L1 expression was significantly increased by monocytes. Less than 1% of monocytes incubated in the absence of TCELNA® product for 24 hours expressed PD-L1 . In contrast, an average of 97.8% of monocytes expressed PD-L1 after 24 hours of incubation with a TCELNA® product (n=4).

### Example 2

### Cell Lines as a Source of Antigen Presenting Cells

Cells from formulated and irradiated TCELNA® product are combined with U937 or THP-1 cells at a 1:1 ratio. Twenty-four and 72 hours later, the culture is harvested and evaluated for PD-L1 expression on U937 or THP-1 cells using flow cytometry. It is expected that PD-L1 expression by U937 or THP-1 cells will increase after incubation with TCELNA® product.

All patents and patent publications referred to herein are hereby incorporated by reference.

Certain modifications and improvements will occur to those skilled in the art upon a reading of the foregoing description. It should be understood that all such modifications and improvements have been deleted herein for the sake of conciseness and readability but are properly within the scope of the following claims.

1. A method of determining the tolerogenicity of a therapeutic agent comprising
a. contacting the therapeutic agent with a test sample comprising test antigen presenting cells in vitro;
b. measuring expression of at least one tolerogenic marker comprising a PD-1 ligand by the test antigen presenting cells contacted with the therapeutic agent, wherein the measuring step occurs within 72 hours after the contacting step; and
c. comparing the expression of the at least one tolerogenic marker by the test antigen presenting cells with an appropriate control;
wherein an increase in expression of the at least one tolerogenic marker by the test antigen presenting cells compared to the control antigen presenting cells determines that the therapeutic agent is tolerogenic.

2. The method of claim 1, wherein the measuring step occurs about 24 hours after the contacting step.

3. The method according to claim 1, wherein the therapeutic agent comprises T cells and the test and control antigen presenting cells are autologous to the T cells.

4. The method according to claim 1, wherein the therapeutic agent comprises T cells and the test and control antigen presenting cells are allogeneic to the T cells.

5. The method according to claim 1, wherein the T cells are reactive to an antigen selected from the group consisting of an allergen and an autoantigen.

6. The method according to claim 5, wherein the T cells are reactive to an autoantigen.

7. The method according to claim 5, wherein the autoantigen is selected from the group consisting of a myelin protein, aquaporin, or platelet glycoprotein

8. The method according to claim 7, wherein the myelin protein is selected from the group consisting of myelin basic protein, proteolipid protein, and myelin oligodendrocyte protein.

9. The method according to claim 1, wherein the PD-1 ligand is PD-L1.

10. The method according to claim 1, wherein the antigen presenting cells comprise monocytes.

11. The method according to claim 1, wherein the antigen presenting cells comprise B cells.

12. The method according to claim 1, wherein the antigen presenting cells comprise dendritic cells.

13. The method according to claim 1, wherein said measuring step comprises incubating the test and control antigen presenting cells with an antibody specific for the tolerogenic marker and measuring the amount of antibody bound to the test and control antigen presenting cells

14. The method according to claim 13, wherein the antibody is labeled with a detectable label.

15. The method according to claim 14, wherein said antibody is detected by flow cytometry.

16. A kit for determining the tolerogenicity of a therapeutic marker comprising an antibody to a tolerogenic marker and an antibody to an antigen presenting cell marker.
